(19) United States
(12) Patent Application Publication
(10) Pub. No.: US 2017/0231816 A1
(43) Pub. Date: Aug. 17, 2017

(54) OPHTHALMIC INJECTION DEVICE AND METHOD

(71) Applicant: Edwin Ryan, St. Paul, MN (US)

(72) Inventor: Edwin Ryan, St. Paul, MN (US)

(21) Appl. No.: 15/435,091

(22) Filed: Feb. 16, 2017

Related U.S. Application Data
(60) Provisional application No. 62/295,937, filed on Feb. 16, 2016.

(51) Int. Cl. A61F 9/00; A61M 19/00

(52) U.S. Cl. CPC A61F 9/0008; A61M 19/00; A61M 2205/3606; A61M 2205/3646; A61M 2205/3368; A61M 2205/13; A61M 2205/0216; A61M 2205/3673; A61M 2205/366; A61M 2210/0612; A61M 2205/502

(57) ABSTRACT

An injection tool and associated methods are shown. Example tools and methods described provide cooling to numb a local area prior to an injection. The local numbing can be used to reduce or eliminate pain associated with the subsequent injection. In selected examples, circuitry and one or more sensors are used to calculate an appropriate contact time of a heat sink with the local region to best provide a desired amount of numbing.

### CLAIM OF PRIORITY

This application claims the benefit of priority to U.S. Provisional Patent Application Ser. No. 62/295,937, filed on Feb. 16, 2016, which is hereby incorporated by reference herein in its entirety.

### TECHNICAL FIELD

This invention relates to devices and methods for giving injections. Specifically, example devices and methods shown are useful in ophthalmic injection procedures.

### BACKGROUND

A number of ophthalmological procedures require injections into a patient's eye. Such a procedure can be painful to the patient, and it is desirable to reduce such pain and/or discomfort in the patient. Topical injections of an anesthetic can be given in a region of the eye prior to an injection into the eye. The injection of a topical anesthetic can reduce the pain involved with the later injection into the eye. However, topical injections require highly skilled technique when injecting into only a topical portion of an eye. Easier and safer tools and techniques for preparing for an injection into an eye are desired.

### BRIEF DESCRIPTION OF THE DRAWINGS

FIG. 1A shows a flow diagram of an example method according to an embodiment of the present invention.

FIG. 1B shows an injection tool according to an embodiment of the present invention.

FIG. 2A shows a heat sink according to an embodiment of the present invention.

FIG. 2B shows another heat sink according to an embodiment of the present invention.

FIG. 3A shows a heat sink and associated components of an injection tool according to an embodiment of the present invention,

FIG. 3B shows the heat sink and associated components from FIG. 3A according to an embodiment of the present invention,

FIG. 4A shows an injection tool in use according to an embodiment of the present invention,

FIG. 4B shows another part of a procedure using an injection tool according to an embodiment of the present invention.

### DETAILED DESCRIPTION

In the following detailed description, reference is made to the accompanying drawings which form a part hereof, and in which is shown, by way of illustration, specific embodiments in which the invention may be practiced. In the drawings, like numerals describe substantially similar components throughout the several views. These embodiments are described in sufficient detail to enable those skilled in the art to practice the invention. Other embodiments may be utilized and structural, or logical changes, etc. may be made without departing from the scope of the present invention.

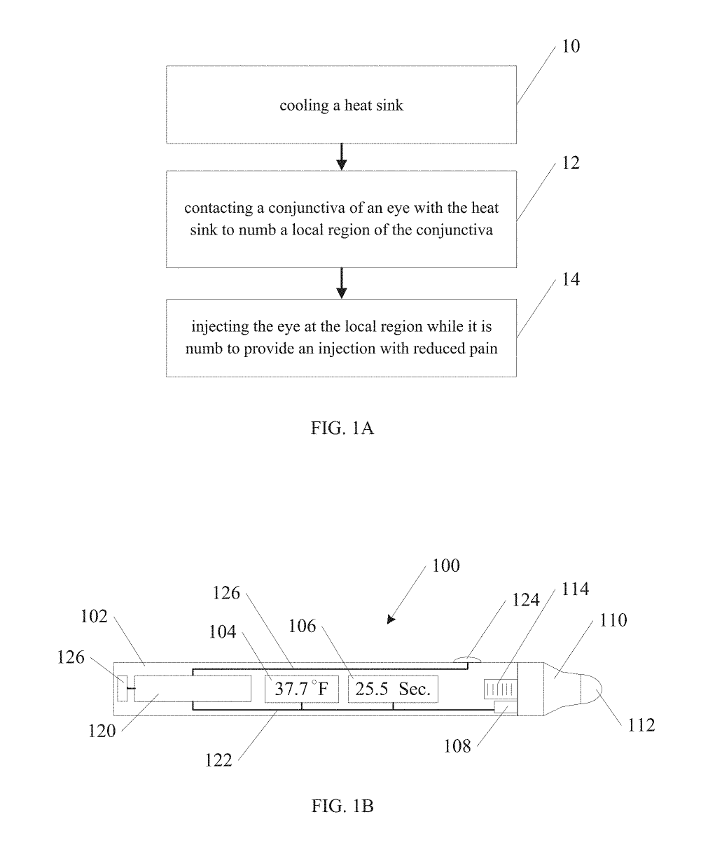

FIG. 1A shows one example of a method of performing an injection procedure into a patient's eye. In operation 10, a heat sink is cooled. In operation 12, the heat sink is applied in contact with a local region of a patient's eye, such as a conjunctiva. Although the conjunctiva is used as an example local region, other regions of a patient's eye are within the scope of the invention. The particular local region of the patient's eye corresponds to a subsequent injection site. Because the heat sink has been cooled in operation 10, the local region of the eye is numbed.

In operation 14, an injection is delivered within the patient's eye with an injection site at the numbed local region. Because the local region has been cooled by the heat sink, the local region is numbed, and the injection is less painful.

FIG. 1B shows one example of an injection tool 100 for use in the method of FIG. 1A. The injection tool 100 includes a handle 102 and a heat sink 110. In one example, the heat sink 110 is removable from the handle 102. In the example shown, the heat sink 110 includes a blunted tissue contact tip 112 sized and shaped for application to a portion of patient's eye, such as a conjunctiva.

In one example, the heat sink 110 includes a continuous smooth surface, with no interruptions or cavities for fluids or contaminants to be trapped. In one example, the heat sink 110 is designed to be sterilized between uses. In such an example, a continuous smooth surface is easier to clean and maintain for multiple uses. An absence of seams, crevices, pits, holes joints, etc. in at least the contact tip 112, and more desirably a majority of the heat sink 110, facilitates ease of sterilization.

In one example, the heat sink 110 is removed from the handle 102 and cooled to prepare for an injection procedure. In one example, the heat sink 110 is placed in a refrigerator or similar cooling environment. In another example, the heat sink is electronically cooled on the handle 102, for example, using a Peltier device, fluid heat exchanger, expanding gas cooler, or other heat exchanger. However, a configuration where the heat sink 110 is removable and cooled in a refrigerator separate from the handle 102 is less expensive to use and manufacture.

In one example, the injection tool 100 of FIG. 1B also includes a thermocouple 108 in thermal communication with the heatsink 110. The example of FIG. 1 further illustrates a temperature indicator 104, although the invention is not so limited. In one example procedure, the heat sink 110 is removed from a refrigerator and is connected to the handle 102 immediately prior to an injection procedure. When using an injection tool configuration that includes a thermocouple 108, a starting temperature of the heat sink 110 is determined. In one example, the starting temperature is further displayed on the temperature indicator 104.

In one example, the user of the injection tool 100 may manually determine if the heat sink 110 is at a proper temperature for the procedure by viewing the temperature indicator 104. In one example, onboard circuitry 120 within the handle 102 may calculate if an acceptable starting temperature exists, using data provided by the thermocouple 108.

In one example, after the heat sink 110 is brought in to contact with a local region of the patient's eye, a temperature of the heat sink 110 is measured over time. In this example, the heat sink 110 can be removed from the patient's eye after a pre-determined temperature of the heat sink 110 is reached. A measured temperature rise of the heat sink 110 correlates to an amount of cooling of tissue in the local region of the patient's eye. By measuring the temperature rise of the heat sink 110, a consistent amount of tissue numbing can be repeatably obtained for an injection procedure.

One of ordinary skill in the art, having the benefit of the present disclosure, will recognize that while a displayed temperature indicator 104 may be useful to a user, it is not required. Temperature calculations may also be performed entirely within the circuitry 120, and once a pre-determined temperature of the heat sink 110 has been reached, the circuitry 120 may trigger an indicator 126 to the user of the injection tool 100 that the heat sink 110 should be removed. Such an indicator 126 may include a light, or sound or other indicator that the heat sink should be removed, and the subsequent injection should be performed.

In one example, a start button 124 is included with the injection tool 100, and is coupled to the circuitry 120. In other examples, a start button 124 is not required, for example where the circuitry 120 determines a start and stop of a numbing procedure using only data collected from the thermocouple 108.

In one example, the injection tool 100 of FIG. 1 also includes a timer. In one example, the timer is included within onboard circuitry 120. The example of FIG. 1 further illustrates a time indicator 106, although the invention is not so limited. In one example, after the heat sink 110 is brought in to contact with a local region of the patient's eye, a time of contact is determined using the timer. In one example a beginning of contact is determined by use of the start button 124. In another example, a start time is triggered by a temperature rise in the heat sink 110 detected by the circuitry 120 using the thermocouple 108.

In one example, the heat sink 110 can be removed from the patient's eye after a pre-determined time of contact with the eye tissue. In one example both time of contact and temperature data from the thermocouple 108 are used to determine when the heat sink 110 should be removed from the eye tissue.

One of ordinary skill in the art, having the benefit of the present disclosure, will recognize that as with the temperature indicator, while a displayed timer 106 may be useful to a user, it is not required. Timing calculations may also be performed entirely within the circuitry 120, and once a pre-determined time of contact has been reached, the circuitry 120 may trigger the indicator 126 so that the user of the injection tool 100 knows that that the heat sink 110 should be removed, and the subsequent injection should be performed.

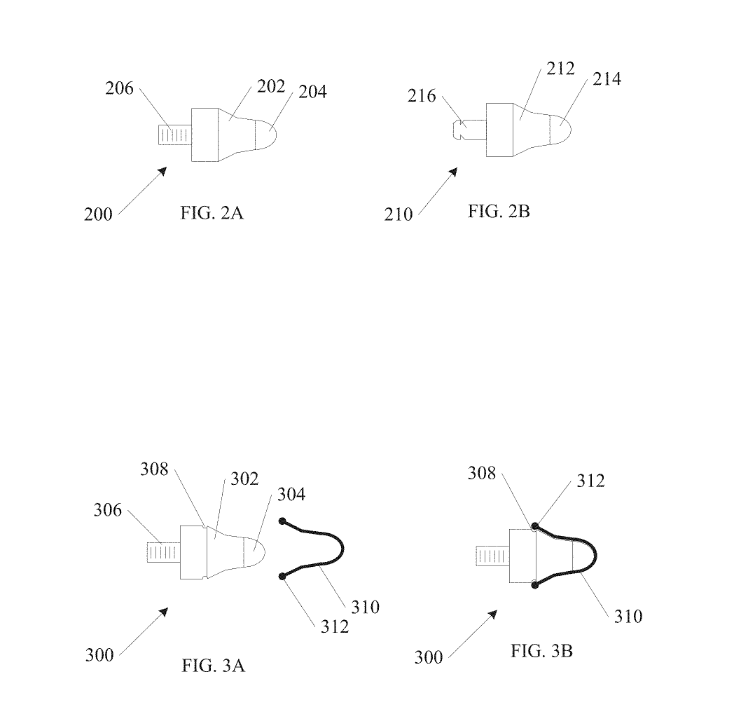

FIG. 2A shows an example of a heat sink 200 similar to the heat sink 110 shown in FIG. 1B. As discussed above, in one example, the heat sink 200 includes a continuous smooth surface 202, with no interruptions or cavities for fluids or contaminants to be trapped. In one example, the heat sink 200 is designed to be sterilized between uses. In such an example, a continuous smooth surface is easier to clean and maintain for multiple uses. An absence of seams, crevices, pits, holes joints, etc. in at least a contact tip 204, and more desirably a majority of the heat sink 200, facilitates ease of sterilization. In one example, the heat sink 200 includes an attachment device 206. The example of FIG. 2A shows a threaded attachment device 206. In the example shown, the attachment device 206 includes male threads that interface with corresponding female threads in the handle 102. However, one of ordinary skill in the art, having the benefit of the present disclosure, will recognize that female threads on the heat sink 200 and male threads on the handle 102 are also within the scope of the invention.

FIG. 2B shows another example of a heat sink 210 similar to the heat sink 200 of FIG. 2A, and 110 shown in FIG. 1B, As discussed above, in one example, the heat sink 210 includes a continuous smooth surface 212, with no interruptions or cavities for fluids or contaminants to be trapped. In one example, the heat sink 210 is designed to be sterilized between uses. In such an example, a continuous smooth surface is easier to clean and maintain for multiple uses. An absence of seams, crevices, pits, holes joints, etc. in at least a contact tip 214, and more desirably a majority of the heat sink 210, facilitates ease of sterilization. In one example, the heat sink 210 includes an attachment device 216, such as a notched attachment device 216. In the example shown, the attachment device 216 is configured to interface with a corresponding tab (not shown) in the handle 102. Similar to the example of FIG. 2A, the notched attachment device 216 is shown as male, however, one of ordinary skill in the art, having the benefit of the present disclosure, will recognize that a female notched attachment may be utilized on the handle 102 are also within the scope of the invention.

FIG. 3A shows another example of a heat sink 300 that may be used with the injection tool 100 of FIG. 1B. Similar to examples described above, the heat sink 300 includes a continuous smooth surface 302, with no interruptions or cavities for fluids or contaminants to be trapped. A contact tip 304 is shown as part of the smooth surface 302. Likewise, the contact tip 304 in the example shown includes a continuous smooth surface. An attachment device 306 is shown to couple the heat sink 300 to an example injection tool, such as the injection tool 100 from FIG. 1B. The attachment device 306 shown includes male threads, however the invention is not so limited. As described above, other attachment devices such as female threads, male or female notched attachment devices, etc. are within the scope of the invention.

FIG. 3A further shows a disposable a 310 that is sized to fit over the contact tip 304. In one example, the disposable barrier 310 is formed from a flexible material, such as plastic. In one example, the disposable harrier 310 is formed from an elastic material such as rubber. In one example, the disposable barrier 310 is formed from a material that conducts heat/cold sufficiently to numb a local region of a patient's eye from the heat sink 30 through the disposable barrier 310. In one example, the disposable barrier 310 is formed sufficiently thin to conduct heat/cold through the disposable barrier 310 and to numb a local region of a patient's eye.

In the example shown, a bead 312 is included at an edge of the disposable barrier 310 to hold the disposable barrier 310 in place on the contact tip 304 during a procedure. In one example, the heat sink 300 includes a corresponding ridge 308 to interact with the bead 312 to hold the disposable barrier 310 in place on the contact tip 304. FIG. 3B shows the disposable barrier 310 with the bead 312 interacting with the ridge 308 to hold the disposable barrier 310 in place on the contact tip 304.

In one example operation, the heat sink 300 is removed from a refrigerator prior to an injection procedure. A disposable barrier 310 is placed over the contact tip 304 of the heat sink 300, and is held in place by the bead 312 and the ridge 308. The disposable barrier 310 provides a sterile contact with a patient's eye, and is discarded after the procedure. Once the local region of the patient's eye is sufficiently numb, as determined by methods discussed above, the injection tool is removed, and the desired injection is delivered in the numbed local region.

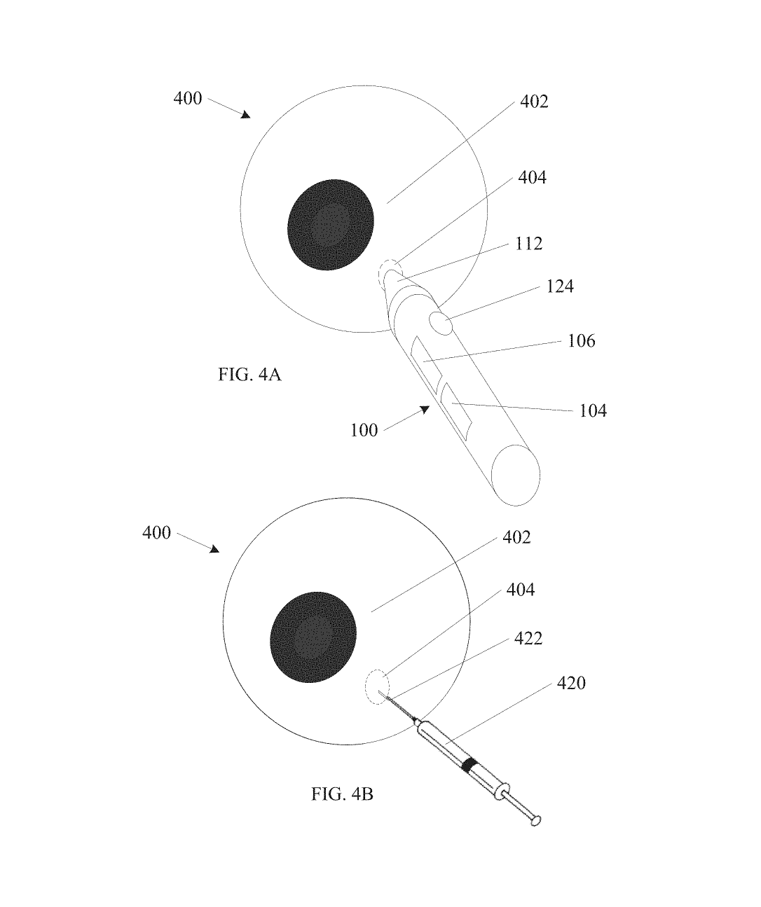

FIG. 4a shows an illustration of an injection tool 100 in use as part of an example injection procedure. The contact tip 112 is shown in contact with a local region 404 of a conjunctiva 402 of a patient's eye 400. In one example, the start button 124 may be pressed after the contact tip 112 first touches the local region 404. As discussed in examples above, the start button 124 may initiate any of a number of possible algorithms carried out by circuitry within the injection tool 100. In one example, a time that the contact tip 112 should remain in contact with the local region 404 is determined by measurement of a thermocouple, that correlates to an amount of cooling of the local region 404. In one example, a time that the contact tip 112 should remain in contact with the local region 404 is determined by a timer that is started by depressing the start button 124. In one example both temperature and time are used to calculate a time that the contact tip 112 should remain in contact with the local region 404. The displays 104, 106 may be used in selected examples to show a user one or more criteria such as temperature of the heat sink, and/or time of contact. In one example, the time indicator display 106 may count down a desired time of contact. In one example, the time of contact is a fixed number. In one example, a time of contact is calculated using data gathered from the thermocouple.

After a desired time of contact between the contact tip 112 and the local region 404 has been determined, and reached, the injection tool 100 is removed from the local region 404. As illustrated in FIG. 4B, after removal of the injection tool 100, an syringe 420 is used to inject a desired active substance within the patient's eye 400. FIG. 4B shows a needle 422 of the syringe 420 inserted at a location within the local region 404. As described above, the prior application of the heat sink to the local region 404 provides sufficient numbing of the local region 404 to reduce or eliminate pain during the injection shown in FIG. 4B.

Although ophthalmic injections are illustrated in the illustrated examples, embodiments of the invention may also be used for injections or stitches in other locations on a patient. Examples include on skin tissue adjacent a wound before stiches are made. Other examples may include utilizing embodiments of the invention before inoculation injections.

As discussed in the background section above, in ophthalmic injection procedures, a first, topical injection of an anesthetic may be used prior to injections within the eye. However, topical injections of anesthetic require a skilled technician or surgeon to properly administer the topical injection. One advantage of using an injection tool as described in examples above includes the relative ease of use, when compared to administering a topical injection.

To better illustrate the method and apparatuses disclosed herein, a non-limiting list of embodiments is provided here:

Example 1 includes a method. The method includes cooling a heat sink, contacting a conjunctiva of an eye with the heat sink to numb a local region of the conjunctiva, and injecting the eye at the local region while it is numb to provide an injection with reduced pain.

Example 2 includes the method of example 1, wherein cooling the heat sink includes refrigerating the heat sink, and removing it from the refrigerator prior to use.

Example 3 includes the method of any one of examples 1-2, further including attaching the heat sink to a handle before contacting the conjunctiva.

Example 4 includes the method of any one of examples 1-3, further including measuring a temperature of the heat sink, and removing the heat sink from the conjunctiva after a pre-determined temperature is reached.

Example 5 includes the method of any one of examples 1-4, further including measuring a time of contact of the heat sink with the conjunctiva, and removing the heat sink from the conjunctiva after a pre-determined time has elapsed.

Example 6 includes the method of any one of examples 1-5, further including measuring a time of contact of the heat sink with the conjunctiva, measuring a temperature of the heat sink, and removing the heat sink from the conjunctiva after a pre-determined combination of time and temperature have been reached.

Example 7 includes an injection tool, comprising a handle, and a heat sink removably coupled to the handle, the heat sink including a blunted tissue contact tip sized and shaped for application to a portion of a conjunctiva.

Example 8 includes the injection tool of example 7, further including a thermocouple coupled to the heat sink.

Example 9 includes the injection tool of any one of examples 7-8, further including a temperature indicator coupled to the thermocouple.

Example 10 includes the injection tool of any one of examples 7-9, further including a timer.

Example 11 includes the injection tool of any one of examples 7-10, wherein the heat sink includes a continuous smooth surface.

Example 12 includes the injection tool of any one of examples 7-11, further including a disposable barrier sized to fit over the blunted tip.

Example 13 includes the injection tool of any one of examples 7-12, wherein the disposable barrier is formed at least partially from an elastic material.

Example 14 includes the injection tool of any one of examples 7-13, wherein the heat sink includes a ridge to hold the disposable barrier in place during use.

Example 15 includes the injection tool of any one of examples 7-14, wherein the heat sink is formed from a metallic material.

Example 16 includes the injection tool of any one of examples 7-15, wherein the heat sink is formed from stainless steel.

Example 17 includes the injection tool of any one of examples 7-16, wherein the heat sink is threadably coupled to the handle.

These and other examples and features of the present infusion devices, and related methods will be set forth in part in the above detailed description. This overview is intended to provide non-limiting examples of the present subject matter—it is not intended to provide an exclusive or exhaustive explanation.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A method, comprising:
cooling a heat sink;
contacting a conjunctiva of an eye with the heat sink to numb a local region of the conjunctiva;
injecting the eye at the local region while it is numb to provide an injection with reduced pain.

2. The method of claim 1, wherein cooling the heat sink includes refrigerating the heat sink, and removing it from the refrigerator prior to use.

3. The method of claim 1, further including attaching the heat sink to a handle before contacting the conjunctiva.

4. The method of claim 1, further including measuring a temperature of the heat sink, and removing the heat sink from the conjunctiva after a pre-determined temperature is reached.

5. The method of claim 1, further including measuring a time of contact of the heat sink with the conjunctiva, and removing the heat sink from the conjunctiva after a pre-determined time has elapsed.

6. The method of claim 1, further including:
measuring a time of contact of the heat sink with the conjunctiva;
measuring a temperature of the heat sink; and
removing the heat sink from the conjunctiva after a pre-determined combination of time and temperature have been reached.

7. An injection tool, comprising:
a handle;
a heat sink removably coupled to the handle, the heat sink including a blunted tissue contact tip sized and shaped for application to a portion of a conjunctiva.

8. The injection tool of claim 7, further including a thermocouple coupled to the heat sink.

9. The injection tool of claim 8, further including a temperature indicator coupled to the thermocouple.

10. The injection tool of claim 7, further including a timer.

11. The injection tool of claim 7, wherein the heat sink includes a continuous smooth surface.

12. The injection tool of claim 7, further including a disposable barrier sized to fit over the blunted tip.

13. The injection tool of claim 12, wherein the disposable barrier is formed at least partially from an elastic material.

14. The injection tool of claim 12, wherein the heat sink includes a ridge to hold the disposable barrier in place during use.

15. The injection tool of claim 7, wherein the heat sink is formed from a metallic material.

16. The injection tool of claim 15, wherein the heat sink is formed from stainless steel.

17. The injection tool of claim 7, wherein the heat sink is threadably coupled to the handle.
